(19) United States
(12) Patent Application Publication
(10) Pub. No.: US 2021/0023315 A1
(43) Pub. Date: Jan. 28, 2021

(54) CONTROLLED-DOSE MEDICINAL LIQUID VAPING DEVICE

(71) Applicant: Remedio Laboratories, Inc., Jenkintown, PA (US)

(72) Inventors: Seth Goldenberg, Elkins Park, PA (US); Eric Christopher Sugalski, Berwyn, PA (US); Christopher H. Scholl, West Chester, PA (US); Andrew Mazzotta, Philadelphia, PA (US); Joseph W Jackson, JR., Wilmington, DE (US); Jonah Younghun Ko, Cambridge, MA (US)

(73) Assignee: Remedio Laboratories, Inc., Jenkintown, PA (US)

(21) Appl. No.: 16/834,953

(22) Filed: Mar. 30, 2020

Related U.S. Application Data
(60) Provisional application No. 62/825,935, filed on Mar. 29, 2019.

(51) Int. Cl. A61M 11/04; A61M 15/00; A61J 7/00

(52) U.S. Cl. CPC A61M 11/041; A61M 15/0025; A61M 2205/8206; A61M 2205/3368; A61J 7/0053

(57) ABSTRACT

A medicinal oil delivery device includes a liquid medicinal oil storage reservoir that stores liquid medicinal oil; a heating element that converts the liquid medicinal oil into vaporous medicinal oil; a delivery device that delivers the liquid medicinal oil to the heating element; and a mouthpiece through which a user can receive the vaporous medicinal oil. The medicinal oil delivery device may include a body that includes the heating element and a removable cartridge engageable to the device that includes the liquid medicinal oil reservoir and mouthpiece.

### BACKGROUND

Medical professionals may use medicinal oils and liquids to treat patients suffering from a range of symptoms. Patients may inhale medicinal compounds using inhalers that vaporize the medicinal liquid for inhalation but delivery of medicinal liquids remains inconsistent and faces challenges such as:
Control of dosage: Controlled target dosing to a high degree of precision. Currently, dosage is controlled by either the user drawing in a breath for an uncontrolled dosage according to their inhalation, or a pressurized delivery that drives a dose into a user's mouth or nose according to a hold time of the pressure applicator and mixture in the medicinal cartridge.
Avoiding the need for entirely custom equipment where off-the-shelf equipment will suffice.
Vaporization/Aerosolization challenges: medicinal liquid must be vaporized (by heat) to create a gaseous mixture for inhalation to maximize bioavailability to the user, but such vaporization may be inconsistent and deliver variable dosing, while also resulting in wasted medicinal oil not vaporized.
Maintaining and minimizing vaporization temperature: In many contexts, the theoretical vaporization temperature of medicinal liquids is greater than 100° C.; maintaining and not exceeding this temperature would result in consistent dosing and energy conservation.
medicinal liquid substrate interaction: In the heating and vaporization, medicinal oil may be exposed to materials that contribute contaminants to the inhaled vapor.
Cycle time length: From the time a medicinal oil inhalation device is turned on until it is ready to use can take inconveniently long.
Device capacity: Some devices use a storage cartridge for the medicinal liquid. Maximizing the number of uses required before cartridge replacement remains a goal.
Reusability: Devices that are not reusable, or that lack reusable components create unnecessary waste and a need to carry multiple devices or components.

The device herein addresses these challenges.

### SUMMARY OF THE EMBODIMENTS

A medicinal liquid delivery device includes a liquid medicinal liquid storage reservoir that stores liquid medicinal mixtures; a heating element that converts the liquid medicinal liquid into vaporous medicinal mixture; a delivery device that delivers the liquid medicinal liquid to the heating element; and a mouthpiece through which a user can receive the vaporous medicinal mixture. The medicinal liquid delivery device may include a body that includes the heating element and a removable cartridge engageable to the device that includes the liquid medicinal liquid reservoir and mouthpiece.

### BRIEF DESCRIPTION OF THE DRAWINGS

The figures, drawings, and presentations attached hereto provide views, graphs, and other images that supplement the description, in particular a description of a breadboard version of the device in which some or all of the components described herein may be used.

The figures summarized below accompany the Detailed Description that follows but do not in any way limit the device to the views therein discussed.

FIG. 1 shows a schematic of a medicinal delivery device described herein.

FIG. 2 shows an illustration of a proposed device.

FIG. 3 shows a cross sectional cutaway view showing the layout for components within the device body.

FIG. 4 shows different views of the removable cartridge.

FIG. 5 shows the operation of the tip, disc, liquid, heating element, and eventual vapor that travels to the mouthpiece.

FIG. 6 shows the convoluted path through to the mouthpiece in one embodiment.

FIG. 7 shows Table 1.

### DETAILED DESCRIPTION OF THE EMBODIMENTS

### 1. Hardware Overview

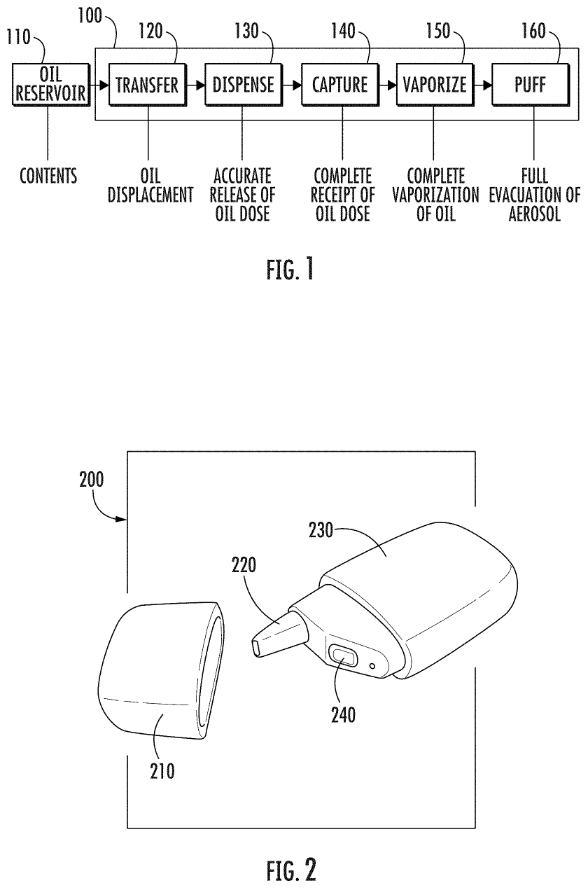

FIG. 1 shows a schematic of a medicinal oil delivery device 100. Although oil, medicinal liquid, medicinal oil, and CBD oil may be referenced herein, it should be understood that the medicinal fluid may be any fluid or fluid mixture where dosage control is a priority. Such a liquid medicine may include a medicine (like an antibiotic, antiviral, THC, CBD, nicotine, etc.) in a solution.

In this schematic, an oil reservoir 110 stores the medicinal oil, which is shown separate from the device 100, although the arrow indicates that they are in fluid communication with each other, and the oil reservoir 110 and device 100 may be integrally or removably connected to one another. The device 100 removes a quantity of medicinal oil from the device 120, and accurately releases/dispenses some of the medicinal oil 130, which is captured 140 for vaporization 150 and finally delivered 160 to a patient. Although some of these steps may be combined (transfer/dispense/capture in particular), this schematic describes an overview of the steps and components required for a functioning device 100.

The target dose of the oil may be any range from 5 to 50 μL, for example targeting 15 μL +10% (13.5 μL-16.5 μL). The dose target of 15 μL is a preliminary target subject to change based on development of both formulation and device output, but can be modified to meet various therapeutic, physical, chemical, or bioavailability demands.

The oil may be vaporized (by heat) to maximize inhaled bioavailability to a user at theoretical vaporization temperatures, for example CBD oil of 170° C., and thus the system may attempt to minimize this temperature within other requirements. The oil/liquid may be a medicine within a solution, such as an antibiotic like vancomycin (which often produces a known localized reaction and thus may be desired to be spread out over the entire surface area of a patient's lungs to prevent this reaction) in water. Because vancomycin and water have disparate melting points and boiling points (above 360 and 100 degrees C. respectively), heating just the water to above 100 C would render the water a vapor but leave the vancomycin unchanged, which allows the water vapor to act as a delivery mechanism for the medicine to the user through the mouthpiece 220. The difference between the delivery solution and medicine may preferably be at least 50 C, but could be less for some medicinal compounds

FIG. 2 shows an illustration of a proposed device 200. The device 200 includes a cover 210, that when removed or opened, exposes a mouthpiece 220 that delivers the vaporized medicinal oil. The mouthpiece 220, along with the cover, medicinal oil cartridge, and other components, may be permanent or removably replaceable depending on the anticipated lifetime of the device 200. The device 200 further includes a body 230 that is hollow but contains the components necessary to store and deliver the vaporized medicinal oil including a CDB oil or other liquid storage reservoir as well as a transfer/dispense/capture element(s) that are activated, along with a vaporizer and delivery mechanism, upon depression of an activation button 240.

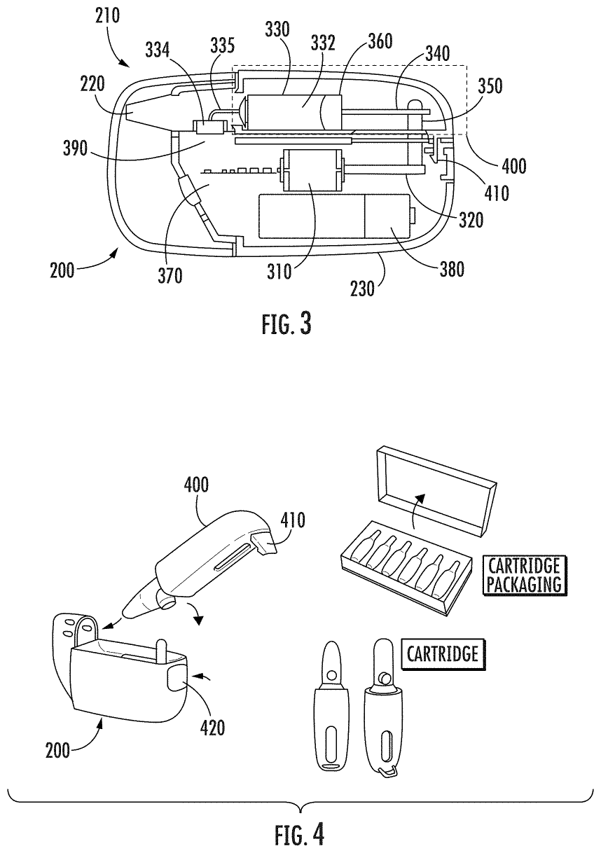

FIG. 3 shows one layout for components within the device body 230, within which there may be a motor 310 with lead screw 320 to drive a compact syringe plunger 330 within the body 230 during dosing. As the motor 310 drives the lead screw 320 threads—which are threaded into the plunger rod 340 bracket 350—the fixed orientation of the plunger bracket 350 forces it to translate along the threads, thus moving the plunger rod 340 and plunger 360 dispensing medicinal liquid 332 stored within the syringe 330. This motor 310 could return to “home” position when a user replaces a cartridge (described later), and its plunger-driving bracket 350 may have an insertion slot that receives a new medicinal oil cartridge.

The device 200 would preferably operate using a device cycle time that minimizes the time required for user (from “On” to finished inhalation). The deliver would preferably be nearly instantaneous with 3 minutes is a target maximum.

The syringe 330 may provide capacity for 50 uses (before disposal or refill/component swap).

The body 230 may include sensors such as: a tilt sensor that notifies the user of an excessive tilting and lock-out the vertical oil drop and/or a beam or vapor sensor that would detect an oil drop (a metric for a dose) or the presence/absence of visible vapor to signal the end of a vaporization cycle. A central control board 370 may control power condition, signaling, and controls for the sensors, motor, heater, dose tracking, etc. including prevention of dispensing if the tilt sensor was at an angle that would hinder dispensing the medicinal liquid.

The power source is shown here as a rechargeable lithium ion battery 380.

The syringe 330 dispenses the medicinal liquid 332 onto a porous disc 334, which allows the medicinal oil to be heated by a heating plate 390 for vaporization and delivery of the vaporized medicinal oil through the mouthpiece 220.

The porous disc 334 may be a vaporization substrate and preferably, the oil would not be exposed to materials at high temperature that could contribute contaminants to vapor. The disc 334 may be made from ceramic, stainless steel, copper or other material suitably chosen for its lack of reactivity with the medicine to be delivered when heated, and the heating disc may be reusable 334.

The heating element for the device 200 may include a porous, stainless steel disc 334 that is heated with a conductive heating element 390 with a preferable porosity that ranges between 5 μm and 20 μm and be of a dimension of, 6.4 mm diameter×1.59 mm thick made from 316L stainless steel. The heater 390 may be replaceable, or just the disc 334, without replacing other body elements.

FIG. 5 shows the operation of the tip 335, disc 334, oil 337, heating element 390 and eventual vapor 338 that travels to the mouthpiece 220. Moving from left to right in sequence, the dispenser tip 335 drops CBD oil 337 onto the substrate disc 334, where the medicinal liquid disperses 337 through the disc 334. The heating element 390 heats the oil 337a turning it into vapor 338 for delivery to the user.

The oil's adhesive and surface tension properties may affect droplet behavior far more than volume. Therefore, the detection of a free-falling droplet of oil and not the displacement value of the syringe pump may be preferable to determine the amount of oil delivered, though both could be possible. The feedback may be in the form of a beam-break or optical sensor, temperature fluctuation measurement in porous substrate, resistive fluctuation measurement across porous substrate, or similar. Drop dispensing may require a level device orientation. This can be achieved with an accelerometer sensor in the device, with device features to provide guidance to correct the orientation (within a specification to be determined) before the dispense can be activated.

In the syringe, straight needle dispensing tips or tapered dispensing tips may be used. Polypropylene appears feasible as a tip material, however an oleophobic material (metallic or not) may likely provide the most consistent dispensing behavior.

The syringe 330 plunger 360, and mouthpiece 220 as well as other components notably including the medicinal liquid 332 may be contained in replaceable cartridges 400 shown in both FIGS. 3 and 4, in which a press-clip engagement 410 (using cartridge release button 420) allows for removal of the cartridge 400 from the body 230. Different cartridges 400 may contain different medicines and be useable with the same body 230, as long as the medicines therein are acceptable to be used together.

The cartridges 400 may include mechanical, RFID, or other identifiers that interact with a controller in the body such that the identifiers relay information about preheating requirements, vaporization temperature, or cycle time to the device 200 such that in user, the heating element 390, motor 310, and other components work together to deliver the optimal dose of the medicinal oil. These parameters could also be controlled by an app remote from the device on a user's phone or similar in communication with the device 200, or they could be set using an app that reads an identification on a cartridge 400 like a QR code before inserting it into the device 200.

The body 230 may house a different heating element, or the heating element disc may be used to preheat the liquid in the cartridge to increase its viscosity.

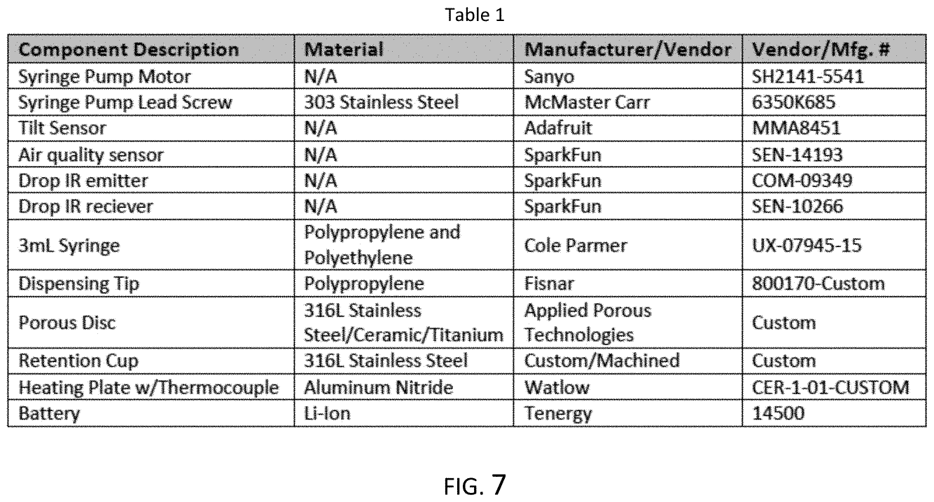

Use of many existing components make it possible to manufacture the above device using for example, components shown in Table 1 shown in FIG. 7.

### 2. Functionality Introduction-Test Case and Designs

The inventors developed a proof-of-concept breadboard that vaporized CBD oil with a high degree of dosing accuracy and repeatability. They sought to vaporize 15 μL of CBD oil with an accuracy of ±10%, repeatedly over 50 doses. The breadboard goal was to have the potential to be turned into a commercial consumer/medical product. After researching the solutions and shortcomings of commercial products in the vaping and general drug delivery space and testing additional systems in the lab, the inventors identified a positive displacement pump technology and a conductive, porous substrate for the dispensing and vaporization system that meets the ±10% accuracy goal.

With a cycle time of approximately 2 minutes 30 seconds, the breadboard system vaporized the specified dose within an error of +2%/−5%. This technology does not appear to be utilized in any other commercial product currently known to the inventors. This meets or exceeds many drug delivery requirements by the FDA, USP and other global regulatory/standards bodies.

### 2.1 Device/User Goals

Both the dispensing and heating may be controlled in a manner such that vaporization is efficient and repeatable per requirements listed below.
Target Dose of Liquid CBD Oil or medicinal mixtures: 15 μL +10% (13.5 μL-16.5 μL). The dose target of 15 μL is a preliminary target subject to change based on development of both oil formulation and device output.
Vaporization (aka: Aerosolization): Oil will be vaporized (by heat) to maximize inhaled bioavailability of the compound to user, inhalation skips first pass metabolism associated with oral delivery.
Vaporization Temperature: Theoretical vaporization temperature of CBD oil is 170° C. and thus the system will attempt to minimize this temperature within other requirements. If the carrier substrate is water, it would be vaporized at 100 C.
Vaporization Substrate: The oil or carrier liquid should not be exposed to materials at high temperature that could contribute contaminants to vapor.
Device Cycle Time: The system design will attempt to minimize time required for user (from “On” to finished inhalation). Near instantaneous is a target goal with 3 minutes is a target maximum.
Device Capacity: System design may provide capacity for 50 uses (before disposal or refill/component swap).
Reusability: Preference is for a reusable device; however, some components may be disposable or swappable.

### 2.2 Operation

The breadboard functioned by delivering a precise drop of CBD oil onto a conductively heated porous substrate, which vaporized the oil. Using before/after weight measurements with a precision laboratory scale, vaporized product was measured to be +2%/−5% of the dropped weight. Error in the total dose was primarily due to oil dispense accuracy (accounting for +3%/−4% of the target weight), although further testing with the refined breadboard improved this dispense accuracy to ±2%. Oil residue left behind on porous substrate after each vaporization contributes about −1% inaccuracy. At the completion of this phase, optimal breadboard test parameters were as follows:
Temperature of porous disc substrate: 300° C.
Porous disc porosity: 10 μm filter size, ˜41% porosity
Porous disc material: 316L Stainless Steel
Porous disc dimensions: 6.35 mm ∅, 1.6 mm thick (dose volume dependent)
Dispense method: Positive displacement with tapered tip and drop detection
Flowrate: 50 μL/minute
User Cycle time: 2 min 30 sec total. ˜1 min pre-heat; ˜1 min 30 sec dispense & vaporization

### 2.2.1 Transfer and Dispense System

A laboratory-grade syringe pump was selected as the method for storing, transferring, and dispensing the oil. Syringe pumps are simple, accurate, and repeatable in transferring small volumes of fluid across varying viscosities using positive displacement. This device also converts well to a finished product design, being used in a variety of commercial and medical products requiring accurate fluid dosing and has the capability to be reloaded with a new syringe-and-heater cartridge for reusability.

Syringe pumps can be programmed for syringes of different capacity, flowrates, and dispensed volumes, making it ideal for breadboard testing. Testing was also widely performed with a positive displacement repeat dispenser—essentially a handheld mechanical syringe pump with pre-set dispensing volumes.

The syringes used in the syringe pump were 3 ml syringes, which enabled the syringe pump to dispense at a higher rate. Luer extensions and a 90° fitting were used to allow proper positioning of the heater plate and substrate for dropping oil. Syringes of 1 mL may also be used in the system, however, may not be optimal due to their higher fluid resistance (from a smaller ID), which limits the flow rate range achievable by the pump motor at this exploratory stage. The syringe pump may provide a stall alarm when fluid resistance is too high for its motor. For reference, 50 doses of a 15 μL requires a capacity of 0.75 mL. It is worth noting, however, that the aspect ratio of the syringe can change, i.e. a shorter, wider syringe may have benefits over a longer, narrower syringe. The primary tradeoffs with these modifications are length of system vs. accuracy of the pump motor (i.e. a larger syringe ID requires a smaller stroke and thus higher motor accuracy), as well as cost for a custom syringe profile if selected.

Dispensing tips, utilized in many fields from electronics manufacturing to drug delivery, were determined to be an important component to the accuracy of the dose. Tip properties (tip shape, diameter, material) are key factors in determining an accurate volumetric dose of the oil due to the oil's small volume and high viscosity. The final breadboard system utilizes a polypropylene, tapered style dispenser tip with Luer lock. These tips are ideal for high viscosity fluids and for reducing the backpressure on the pump in contrast to needle-style tips with a long, small inner diameter lumen. The oil droplet was dispensed and allowed to freefall ˜30 mm onto the heating sub-system.

One challenge of dispensing a viscous fluid in such a small volume is that the oil's adhesive and surface tension properties tend to affect droplet behavior far more than volume. Therefore, testing was performed to identify the reliability of “drop-dosing”, wherein a dose is determined by tip geometry, fluid viscosity, and detection of a free-falling droplet of oil and not the displacement value of the syringe pump. Testing on this method is covered below and appears to be a promising method of dose control, which would require additional sensing capabilities, and could also address other concerns with accurate dosing.

Heat may affect a precision scale's accuracy, so it was not practical to measure each dispensed weight and vaporize it while receiving a live readout on the scale. Additionally, the value of this real-time weight data did not prove to be critical to accuracy targets. Therefore, the dispensing system was verified for accuracy before running final experiments on vaporized dose accuracy. Performance was assumed to be similar between these two experiments due to the environmental and operating conditions of dispensing system testing being identical in both cases (primarily being exposed to heat from the heating sub-system).

Testing in this phase began with dispensing methods, focusing on syringes, pipettes, and repeat dispensers. A precision analytical scale, capable of measurements to 0.0001 g, was used for all weight measurements in the project as a means of confirming the volume dispensed.

The tip touch-off method was employed in subsequent bench testing to tighten the dispense precision. The dispensing tip remained touching the fluid drop on the porous disc throughout the dispense cycle, allowing additional fluid on the tip's outer diameter to wick with the dispensed fluid. This prevented the buildup and high doses shown above. Average weight was 13.9 mg, with a +3%/−4% error.

A repeatable, accurate volume of dispensed oil is valuable to total dose % calculations and is the first major contributor to inaccuracies. After many doses, there may be a certain uptick in delivered doses and a return to normalcy. This phenomenon appears to be attributed to the adhesive behavior of the oil, which clings to the outside of the dispensing tip. Buildup over several cycles (in this case 12) can subsequently produce higher doses. Repeat dispenser manufacturers recommend touching the tapered tip to vessel walls during dispensing to eliminate this buildup on the tip for an accurate dosing. However, in this particular application this practice is difficult for a commercial product—touching off the dispenser tip would be a challenge in this compact, high temperature, automated system. Therefore, other methods were explored to reduce this effect.

As further testing moved to the syringe pump system, straight needle dispensing tips were used initially, followed by a tapered dispensing tip style. The weights of these dispenses were higher than repeat dispenser testing, likely due to the differing tip geometry between the two types of systems and the effect that had on the oil's adhesion to the tip. This highlights the importance of tuning this tip geometry for target dose volume/weight and oil viscosity/adhesion properties.

Through these dispensing tests with the syringe pump and the repeat dispenser, there were often discrepancies between the dispensed volume readout and the mass of the actual oil that was dropped. This appears to be due to uncontrollable accumulations of oil on the tip, producing weight cycling or randomization, as discussed above.

Therefore, in the case of the syringe pump, a change was made to the drop-dose method, whereby a single, freefalling drop was the metric for a dose instead of a metered displacement. While in a commercial product this would require an additional means of sensing a single drop in a discrete (yes/no) method, testing indicates that it will produce an acceptable dose accuracy.

Additionally, it could account for other inconsistencies between doses (such as air bubbles forming, unpredictable oil wicking, etc.) that a metered dispensing method would be unable to address. A flowrate of 50 μL/minute was used in all tests, however this may be of little consequence if the drop- dose method is employed, and dispensing can begin during heater warm-up, effectively adding no time to the full cycle with a wide range of flowrates.

A syringe pump and the tapered dispensing tip have produced a consistent weight of approximately 16.8 mg, ±2%. This was achieved using the drop-dose method discussed above. A 16-gauge dispensing tip was used, with the tip trimmed back until there was a 1.6 mm ID.

Oil has risk of coming out of solution during colder storage temperatures (shipping, left in car, etc.). An oil formulation may become more stable with dilution or other additives, at risk of changes to inhaled vapor. It may be costly (both time of cycle and expense) to provide a corrective solution to this issue within the device, i.e. a heater which brings syringe oil to a programmed temperature before dispensing.

Oil or other liquid volume targets have been based on initial metric. Drop-dispense optimization and other dispensing or heating design factors may point towards a modification to the dose volume/weight ratio.

If the drop-dose method is proved to be technically viable over positive displacement, tight control on viscosity of formulation may be required to produce drops of consistent weight. It will be a challenge to account for other manufacturer's oils in the same device (precision can be expected, but not an accurate dose across different oil types).

Dose accuracy values detailed in the testing assume a homogenous oil solution. The residue by weight therefore assumes there is CBD isolate equally distributed in the residue, however further testing on the residue would confirm the contents and address this question. For example, it is possible that a 1% residue only contains 0.5% of the equivalent CBD (the rest being primarily MCT oil or additives), thus increasing the delivered CBD. Alternatively, perhaps the residue contains more CBD than expected and would reduce the dose delivered.

A tapered dispensing tip is recommended for the application due to its lower hydraulic resistance, and potentially more consistent behavior regarding freefall drop size. Optimizations remain once oil volume target and formula is finalized.

Polypropylene appears feasible as a tip material, however an oleophobic material (metallic or not) may likely provide the most consistent dispensing behavior. Due to the small thermal mass of the Watlow heater used in testing, even with a distance of <25 mm from heater to dispensing tip during heating, the polypropylene tip displayed no deformation. However, temperature limits and proximity of dispensing tip must be considered in material selection and design.

The recommended dispensing method is the drop-dose method in which a free-falling drop is considered a dose. This method can produce consistent weights, is achievable with a syringe pump system, and addresses challenges of the conventional syringe-pump method.

The disadvantage of the conventional syringe pump method (syringe plunger displacement closes the feedback loop for how much oil has been dispensed) is that if the dose in the dispensing tip were to face an unpredictable change (i.e. the device is dropped and produces an air bubble, dispensing tip residual oil stack-up occurs, as seen in testing, etc.), a subsequent displacement of the syringe may produce a missed or double-dose. Expressed another way, the “zero” point is subject to change at the dispensing tip and the syringe pump cannot account for this.

The drop-dose method for identifying a dispensed target volume may need to be assessed further. By tightly controlling oil and dispensing tip properties, early testing has shown that permitting a slowly dispensed drop to freefall from the dispensing tip can produce a consistent weight. The feedback loop may help identify a method of confirming a drop has fallen in the device (beam-break or optical sensor, temperature fluctuation measurement in porous substrate, resistive fluctuation measurement across porous substrate, etc.).

Drop dispensing may require a level device orientation. This can be achieved with an accelerometer sensor in the device, with device features to provide guidance to correct the orientation (within a specification to be determined) before the dispense will be activated.

### 2.2.2 Capture and Vaporize System

Another part of the dosing system is how the CBD oil is captured and vaporized. Commercial products that use oil vaporization as a delivery method require a method of transferring the oil from its reservoir to its heating source. In most commercial devices this is done passively with capillary action of a wicking material. Typical vape pens use fiber wicks, wrapped by a resistive heating coil (Kanthal or Nichrome). Vaporization of the oil at the heater area results in a constant capillary draw of oil that flows from the reservoir through the wick. While an array of technologies exists for vaporizing oils and waxes for inhalation, the wick and coil style system appear to be the only type that does not require single dose loading by the user.

One of the primary drivers behind design selection for the heating sub-system was the coil's material effects on the vapor. Research has shown that the coil materials (Kanthal, Nichrome, etc.) used in the majority of vape pens can contribute toxic levels of various metals to the vapor, which may be inhaled by the user. This is probably because coil materials are optimized for efficient heat transfer and low cost, not vapor purity. While this vape-contaminants issue may not have reached a head yet, it is now being discussed in the press and online forums and may snowball and then necessitate a better solution in the marketplace.

The device herein may take a different approach. The heating sub-assembly for the design may be a porous, stainless steel disc that is heated with a conductive hotplate. These discs are very inexpensive and commercially used in filtration or bubbling applications. For the breadboard, optimal porosity ranges between 5 μm and 20 μm. Off-the-shelf discs used here are 316L stainless steel, 6.4 mm diameter×1.59 mm thick. In this configuration, the disc serves as an all-in-one wick and heater substrate.

By combining the function of wick and heater, the porous disc provides a capture substrate and heater solution in several ways:
Dispensing of oil onto the porous substrate captures and prevents the oil from its natural proclivity of migrating towards colder surfaces, a phenomenon observed in a variety of early testing with smooth substrates.
The porosity of the disc provides a high ratio between substrate surface area to oil. This high- contact ratio delivers fast heat transfer and minimizes the vaporization time of the oil, which appears to reduce residue and benefit usability.

There are many substrate material options aside from 316L stainless steel. Other viable material candidates that can be fabricated the same way include ceramic, nickel, and titanium alloys. 316L SS was selected due to its off-the-shelf availability, high corrosion resistance, and use in a variety of medical applications and cookware, where oils are commonly vaporized (though not intentionally inhaled). Similar considerations should be made when exploring alternative disc materials.

Optimizing vapor release—the ease of vaporization and release from the substrate—was found to have an impact on vaporization time. A porous substrate permits a variety of vaporous exit paths, as opposed to a drop of oil on a flat heated surface.

The substrate shape need not be a disc, necessarily, though it is referred to as a disc herein because that has been found to be preferable and available. The manufacturing process of these porous metals/ceramics enables a wide range of sizes and shapes including convex, concave, or more complex shapes. Shape optimizations may be driven by final dose size, heat transfer, fluid wicking, and vapor release profiles.

A variety of exploratory testing was performed early on in this project to identify viable means of vaporization of the CBD oil. Categorically these were limited to conductive and convective methods, with conduction being the far more favored method after some initial tests.

The heating device throughout testing was originally a laboratory heat plate and subsequently an off-the-shelf but customizable aluminum nitride micro-heater plate from Watlow. The Watlow ULTRAMIC unit was powered with 48 V, had a maximum wattage of 200 W, and included a K-type thermocouple embedded in the center. In both cases, the substrate (within its aluminum or stainless-steel cup) sat directly on the heating devices. There was no pressure or thermal interface material used—these would be intended in a commercial device and would improve temperature distribution and heating efficiency.

As described above, a heated, porous disc was used as the vaporization substrate. Aside from the qualitative testing in which the oil was observed to be captured within the porosity of the disc (a major challenge of heating oil and a primary benefit of the porous disc), testing was performed to confirm that vaporization was complete within specification. This test series was performed on the benchtop setup which used a laboratory grade hotplate, upon which a lightweight aluminum sample cup held the porous disc.

During vaporization testing in which a range of disc porosities were used as vaporization substrates, the disc temperature was 300° C., using the repeat dispenser to drop a 15 μL dose. This enabled subsequent cooling and weighing of the weight change in the aluminum cup/porous disc combination, performed every 10 cycles for 50 cycles. Note that each data point is a cumulative gain (in %) of that data point's previous 10 cycles. The total % over 50 cycles for 10 μm, 60 μm and 100 um was 0.5%, 0.8%, and 1.3%, respectively.

An interesting point in this % figure of weight gain is that this is a % residue by weight of the entire CBD oil content. This is not to say that an equivalent % of CBD is left behind—more testing is required of the either the vapor produced or the residue. Additionally, the residue left behind during heating is also exhibited with MCT oil alone, the largest contributor to the CBD oil makeup, suggesting MCT oil may largely be responsible for the residue.

Visually, residue was visible and while the visual change is drastic, this still accounted for only 1.5% change in weight. Additionally, of note are the rings of residue left behind. This residue had little or no apparent impact on these results, however it is worth noting that subsequent effects were seen in the retention cup scenario in the refined breadboard, where this residue could accumulate in such a way as to trap vapor and slow vaporization time.

Cessation of vapor cloud generation was reliable as a stopping point in that there was no significant weight change after vapor production has stopped. Previous tests had been run where the oil was dispensed onto a room temperature disc, and then the disc in its aluminum cup was placed on the hotplate. The vaporization times below performed on a preheated disc which substantially reduced the vaporization times. Because this directly reduces the required time window in which a user must inhale the vapor, this preheat is recommended.

Variations on vaporization time are subject to a variety of factors. A change in the exhaust duct (actively pulling air) position appeared to have an effect, in that a close position and stronger draw reduced vaporization time. In the data, the position change from close to further only occurred on 10 μm and 100 μm testing, after the tenth cycle test. This was an incidental data point that was not being specifically tested.

Further testing was performed on the refined breadboard system to confirm the effects of vapor release mitigation. Positioning the exhaust duct closer to the vaporization location from 12″ to 4″ had a significant effect on the vaporization time, particularly by slowing the rate of increase as the porous disc becomes potentially more occluded by residue. The change in time is most notable in these tests; the actual vaporization time is overall higher than the earlier tests due to the retention cup geometry not being optimized for vapor release at time of testing—and was not the focus of the results.

One theory behind the vaporization time reduction is that by evacuating the air around (and likely within) the porous disc, vapor pressure within the disc dropped and provided less resistance to vapor generation. Thermal effects were also considered as a source for this change, but it would be expected that localized, higher air evacuation around the disc (i.e. the duct is closer) would slow the vaporization time by cooling the disc.

Taking advantage of this airflow effect is viable in a commercial product. For example, the user could be instructed to inhale as soon as vapor production starts—this would be the simplest solution from a hardware standpoint. Alternatively, assistive convection or a larger chamber around the heating system could produce similar benefits, though with challenges.

There are, however, also optimizations to be made to the porous disc (or substrate) itself. Original breadboard testing, in which a porous disc sat openly on its aluminum sample cup, performed significantly better regarding vaporization time than when the porous disc sat within its retention cup. Additionally, vaporization testing in the retention cup caused bubbling of the oil and slight uplift of the porous disc (a slip fit into the cup) due to the vapor pressure building and releasing. This appeared to be due to the additional surface constraint that the cup provided. These observations identify a key factor in the design iterations to be made on the porous substrate, in that as much surface area is made available to vapor release, but also permits a way to capture residue flow and interchangeability of the puck and retention cup subassembly.

Research performed into vapor contamination through typical heater coil materials may be subject to further research which may corroborate or challenge present results which indicate a health risk. While the substrate material may be subject to further research in this particular way, a porous substrate as recommended here still addresses the issue of dosing, in that it can more readily assure that all dispensed oil is vaporized. This is in contrast to a fiber wick and coil, where—due to far less efficient heat transfer—vaporization time would likely increase.

Efforts may be made to maximize the available surface area of the oil to the outside surface of the substrate. For example, a thinner, wider disc would make oil throughout the substrate face lower resistance to escaping the disc as vapor and may accelerate vaporization time. Additionally, allowing lateral disc exposure may increase the vapor release rate, as was observed in testing. A variety of geometries may be possible here and require further comparative testing.

Porosity of the disc in this reports' experiments is based on the manufacturing process of the off-the-shelf materials acquired. Several manufacturing methods exist for porous metals or metal foams. These styles of porosity can be tightly controlled if necessary and are worth investigating on the basis of cost and/or thermal and vapor release performance.

Thermal optimizations with the porous substrate may be focused on ensuring smooth and strong contact between the substrate and its retention cup in their final forms. Therefore, the retention cup may have a very smooth surface finish on all heater/substrate contact surfaces and the porous substrate be pressed into the cup. Thermal interface materials will likely interfere with wicking and vapor production.

User feedback should be collected at some point to determine the acceptable device cycle time for the target population. This will drive custom heater design (geometry, heater circuit design) and subsequently power requirements and power source (battery) selection.

Following porous substrate and retention cup design, specifications can be further discussed with heater manufacturers (i.e. Watlow) to identify geometry and power requirements. Due to the temperature range, requirements for a non-contaminating heat source, geometric stability to ensure good thermal contact, and relatively high thermal conductivity, a ceramic (Watlow ULTRAMIC was Aluminum Nitride) appears most promising as the heater material.

The ideal temperature for vaporization has not been finalized. Testing targets here have been 300° C. for the porous disc, however 400° or even 500° would likely speed up vaporization time. It can also be reduced to 100 degC based on the melting temperature of the target compound. This wide range of temperatures and ability to precisely control lends the device to significant number of compounds for delivery to patients via inhalation. Risks of higher temperatures which should be assessed include burning of oil, higher chance of deposits into vapor from substrate (requires vapor testing), higher residue, higher power requirements, and better performing thermal insulation in a finished device.

### 2.2.3 Puff System

Due to the focus on a novel vaporization system as the source of dose control, only basic testing was performed with a puff (or inhalation) system during early benchmarking. A syringe was connected to various vape products and drawn in these cases. A weight of these syringes could be taken; however, buoyancy of the vapor may produce errors in these measurements and a more reliable data point would be weight of the heating sub-system before and after vaporization.

A range of puff concepts were considered by exploring market solutions and other methods of air flow control. Additionally, the puff system was considered for the primary dose control method, via flow measurement (user inhalation volume), but ultimately it was deemed more complicated due to the challenges of vaporization consistency. While air flow during inhalation could be monitored in such a way as to measure the volume inhaled, it would be very difficult to ensure consistent CBD content in a given volume of vapor. Therefore, controlling the dose of CBD liquid that is fully vaporized appears to be a more efficient method.

The puff system therefore could likely be a simple open pathway, directly connected to the vaporization zone, through which the user would inhale the vapor. To prevent vapor escape before inhalation, a one-way valve may be required near the mouthpiece and on a specifically located inlet to induce evacuation of the vapor in the vaporization zone. Another alternative (or addition) is a specially designed tortuous path which minimizes chance of vapor escape without the negative pressure induced by inhalation.

Additionally, inhalation during the vaporization process has been seen to yield quicker vaporization times and possibly lower residue.

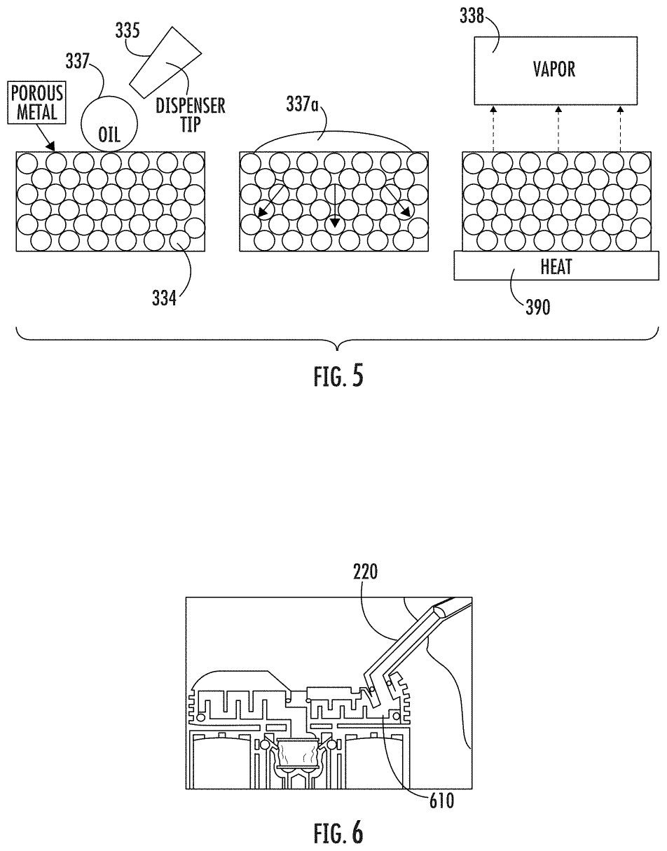

A number of design factors may be considered both in this inhalation pathway and in the vaporization system. An overly short path runs the risk of a vapor that is too hot to be inhaled, and is a problem addressed by many products in the market, typically with a tortuous air pathway or one with passive cooling geometry. It is possible, given the very small volume of oil being vaporized, a short path with minimal cooling effect is viable—in contrast to a solution to the convective heating displayed in FIG. 6.

An overly tortuous inhalation path may provide more opportunity for oil to condense on surfaces and reduce the dose inhaled by the user. Because testing has indicated that faster vapor evacuation from the porous disc helps to reduce vaporization time, this implies the opposite (a small, non-turbulent vapor chamber) would have a negative effect. All of these factors should be considered so that between vaporization and inhalation there is a minimal loss of product.

The vapor chamber around the heating system does not have a finalized volume requirement. Further testing after optimization of the heating system should be performed to identify the appropriate volume to capture vapor without having the ensuing pressure increase vaporization time. A passive cooling channel, as mentioned previously, may be required to cool vapor on its way to inhalation.

As is seen with nebulizers and other inhalation drug delivery devices, it may be preferable for dose delivery that the user follow provided instructions, specifically regarding when and how long to inhale after device activation. It is not unreasonable to expect the same requirements in this device, and this could help reduce vaporization time by having the chamber be exhausted at set intervals. Forced convection (by a fan or other means) does not appear viable due to challenges of capturing this moving vapor.

Sensors exist which are capable of detecting vapor (or its absence). This may be a viable option for detecting vapor production completion to end a heating cycle. These could be air quality/particle, optical, or beam-break sensors.

While the invention has been described with reference to the embodiments above, a person of ordinary skill in the art would understand that various changes or modifications may be made thereto without departing from the scope of the claims.

We claim:

1. A liquid vaporization medicinal delivery device comprising:
a liquid reservoir that stores a medicinal liquid to be vaporized;
a heating element that converts the medicinal liquid into vapor for inhalation by heating the medicinal liquid;
a delivery device that delivers the medicinal liquid in a predetermined amount to the heating element; and
a mouthpiece through which a user can receive the vapor.

2. The device of claim 1, wherein the heating element comprises a disc that is heated by a conductive hotplate.

3. The device of claim 2, wherein the disc is replaceable.

4. The device of claim 2, wherein the delivery device includes a syringe that includes the liquid reservoir and a motor that drives the syringe to deliver the medicinal liquid.

5. The device of claim 2, wherein the disc is a porous disc.

6. The device of claim 5, wherein a porosity of the disc ranges between 5 μm and 20 μm.

7. The device of claim 5, wherein the medicinal liquid disperses through the porous disc before the heating element converts the medicinal liquid to vapor.

8. The device of claim 2, wherein a sensor detects an angle of the medicinal delivery device relative to ground, and relays the angle to a controller that can prevent delivery of the medicinal liquid based on the angle.

9. The device of claim 1, further comprising:
a body that comprises the heating element; and
a removable cartridge engageable to the body that includes the liquid reservoir and mouthpiece.

10. The device of claim 9, wherein before removal of the cartridge from the body, a motor that drives delivery device returns to a home position.

11. The device of claim 9, wherein the removable cartridge engages the body in a press clip engagement.

12. The device of claim 9, wherein the cartridge includes an identifier that conveys information about the medicinal liquid to a controller in the body.

13. The device of claim 12, wherein the information is selected from a group consisting of: vaporization temperature, and cycle time; and wherein the controller executes corresponding controls within the medicinal delivery device based on the information.

14. The device of claim 9, wherein an application remote to the device communicates information is selected from a group consisting of: vaporization temperature, and cycle time; and wherein a controller executes corresponding controls within the medicinal delivery device based on the information.

15. The device of claim 1, further comprising a body that comprises the heating element; and
a removable cartridge engageable to the device that includes the liquid reservoir and mouthpiece.

16. The device of claim 1, wherein the delivery device includes a motor that drives a syringe that includes the liquid reservoir.

17. The device of claim 16, wherein the motor includes a lead screw that drives a plunger on the syringe to release an amount of medicinal liquid.

18. The device of claim 17, wherein the amount is controlled by a controller.

19. The device of claim 1, wherein the delivery device is activated upon depression of a button by a user.

20. The device of claim 1, wherein the device includes a sensor that detects a drop of medicinal liquid.
